(19) United States
(12) Patent Application Publication
(10) Pub. No.: US 2024/0315869 A1
(43) Pub. Date: Sep. 26, 2024

(54) ITCHING RELIEVING APPARATUS AND OPERATING METHOD THEREOF

(71) Applicant: Shenzhen Chuancheng Technology Service Co., Ltd, Shenzhen (CN)

(72) Inventors: Chu Gong, Shenzhen (CN); Weinong Xu, Shenzhen (CN); Anning Chen, Shenzhen (CN); Ying Wei, Shenzhen (CN)

(21) Appl. No.: 18/195,968

(22) Filed: May 11, 2023

(30) Foreign Application Priority Data
Mar. 24, 2023 (CN) 202310352009.8

(51) Int. Cl. A61F 7/00; A61F 7/02

(52) U.S. Cl. CPC A61F 7/007; A61F 7/02; A61F 2007/0052; A61F 2007/0073; A61F 2007/0077; A61F 2007/0284

(57) ABSTRACT

Embodiments of the present invention disclose an itching relieving apparatus and an operating method thereof. The itching relieving apparatus includes a temperature control unit, a heating element, and a blue LED unit; the temperature control unit is configured to: supply power and start the blue LED unit and receive a feedback resistance coefficient corresponding to a current temperature of the heating element; after controlling the heating element to heat up to a target temperature, power on and off the heating element according to the feedback resistance coefficient to keep the temperature of the heating element stable; the heating element is configured to supply heat; and the blue LED unit is configured to start working under the power supply of the temperature control unit, and stabilize a corresponding power supply. By implementing the itching relieving apparatus provided by the present invention, the itching relieving effect on patients is improved.

### CROSS REFERENCE TO RELATED APPLICATIONS

The present application claims the benefit of Chinese Patent Application No. 202310352009.8 filed on Mar. 24, 2023, the contents of which are incorporated herein by reference in their entirety.

### TECHNICAL FIELD

The present invention relates to the technical field of itching relieving equipment, and in particular to an itching relieving apparatus and an operating method thereof.

### BACKGROUND

Mosquito bites make it difficult for people to fall asleep, and also spread diseases. However, it is not easy to completely exterminate mosquitoes, so the mosquito bites are unavoidable. When mosquitoes bite, female mosquitoes will secrete an organic acid similar to an anticoagulant in saliva, which prevents blood from clotting. Thus, the mosquitoes are capable of sucking the blood smoothly. This organic acid, i.e. formic acid, has a chemical component of HCOOH. This substance may cause anaphylactic reaction on skins, such as redness or swelling, itching, pains, and other symptoms.

In an existing itching relieving apparatus, a heating and electric heating element, which is directly used for contacting with patients' skin, is provided on an end part, and sites needing itching relieving are warmed by using the heating and electric heating element so as to achieve the aim of relieving itching. The heating and electric heating element of this product only has a single heating function and uses ordinary dry cells. However, the energy density of the dry cells is low so that the temperature of the heating and electric heating element is not uniform and instable, resulting in a poor itching relieving effect and the like on patients.

Therefore, it is necessary to design a new itching relieving apparatus, which can realize uniform and stable temperature of the heating and electric heating element and improve the itching relieving effect on the patients.

### SUMMARY

A technical problem to be solved by the present invention is to provide an itching relieving apparatus and an operating method thereof.

In order to solve the above technical problems, the purposes of the present invention are achieved by the following technical solutions: An itching relieving apparatus is provided, including a temperature control unit, a heating element, and a blue LED unit; the temperature control unit being configured to: supply power and start the blue LED unit and receive a feedback resistance coefficient corresponding to a current temperature of the heating element; after controlling the heating element to heat up to a target temperature, power on and off the heating element according to the feedback resistance coefficient to keep the temperature of the heating element stable; the heating element being configured to supply heat; and the blue LED unit being configured to start working under the power supply of the temperature control unit, and stabilize a corresponding power supply.

A further technical solution is as follows: the temperature control unit includes a main control chip U4, a thermistor R29, and a resistor R25; the main control chip U4 is connected to the thermistor R29 and the resistor R25, respectively; and where the thermistor R29 is configured to detect the current temperature of the heating element and form the corresponding feedback resistance coefficient.

A further technical solution is as follows: the heating element includes an aluminum oxide substrate heating sheet J11, the aluminum oxide substrate heating sheet J11 being connected to the main control chip U4 through a control chip U2.

A further technical solution is as follows: a power supply unit is further included, the power supply unit being configured to supply power to the temperature control unit, the heating element, and the blue LED unit.

A further technical solution is as follows: the blue LED unit includes a voltage regulator module, a blue light control module, and an LED blue light, the blue light control module being connected to the main control chip U4 and the power supply unit, respectively; and the voltage regulator module being connected between the power supply unit and the blue light control module.

A further technical solution is as follows: a start unit is further included, the start unit including a start key S1 and a start key S2 and the start key S1 and the start key S2 being connected to the main control chip U4.

A further technical solution is as follows: a housing is further included, the housing being internally provided with a main board and the temperature control unit being provided on the main board.

A further technical solution is as follows: an upper end face of the housing is downward recessed to form a groove, the LED blue light is placed in the groove, and a transparent cover caps over the LED blue light.

A further technical solution is as follows: one end of the housing far away from the LED blue light is connected to a cover body through a buckle structure.

Additionally, a technical problem to be solved by the present invention is also to provide an operating method of an itching relieving apparatus, including:

in a case that the itching relieving apparatus works, after a temperature control unit controls a heating element to heat up to a target temperature according to a feedback resistance coefficient corresponding to a current temperature of the heating element, powering on and off the heating element according to the feedback resistance coefficient to keep the temperature of the heating element stable; and providing stable voltage through the temperature control unit and a blue LED unit to ensure that a peak wavelength of blue LEDs is output without loss.

Compared with the prior art, the present invention has the following beneficial effects: According to the present invention, the temperature control unit, the heating element, and the blue LED unit are arranged and the temperature control unit is used for controlling the temperature of the heating element in real time; when the temperature of the heating element reaches the target temperature, the temperature of the heating element is kept stable by powering on and off within a specified time period to realize uniform and stable temperature of the heating and electric heating element and provide stable voltage for the LED blue light, thereby improving the itching relieving effect on the patients.

The present invention is further described below with reference to the accompanying drawings and the specific embodiments.

### BRIEF DESCRIPTION OF THE DRAWINGS

To describe the technical solutions in the embodiments of the present invention more clearly, the following briefly describes the accompanying drawings required for describing the embodiments. Apparently, the accompanying drawings in the following description show some embodiments of the present invention, and a person of ordinary skill in the art may still derive other accompanying drawings from these accompanying drawings without creative efforts.

FIG. 1 is a schematic block diagram of an itching relieving apparatus provided by an embodiment of the present invention.

FIG. 2 is a specific schematic circuit diagram of a temperature control unit of an itching relieving apparatus provided by an embodiment of the present invention.

FIG. 3 is a specific schematic circuit diagram of a heating element of an itching relieving apparatus provided by an embodiment of the present invention.

FIG. 4 is a specific schematic circuit diagram of a start unit of an itching relieving apparatus provided by an embodiment of the present invention.

FIG. 5 is a specific schematic circuit diagram of a voltage regulator module of an itching relieving apparatus provided by an embodiment of the present invention.

FIG. 6 is a specific schematic circuit diagram of a blue light control module of an itching relieving apparatus provided by an embodiment of the present invention.

FIG. 7 is a specific schematic circuit diagram of an LED blue light of an itching relieving apparatus provided by an embodiment of the present invention.

FIG. 8 is a specific schematic circuit diagram of a 5 V charging input module of an itching relieving apparatus provided by an embodiment of the present invention.

FIG. 9 is a specific schematic circuit diagram of an input protection module of an itching relieving apparatus provided by an embodiment of the present invention.

FIG. 10 is a specific schematic circuit diagram of a charging IC of an itching relieving apparatus provided by an embodiment of the present invention.

FIG. 11 is a schematic three-dimensional structural view of an itching relieving apparatus provided by an embodiment of the present invention.

FIG. 12 is a schematic exploded structural view of an itching relieving apparatus provided by an embodiment of the present invention.

Reference numerals in the figures:

10: temperature control unit; 20: heating element; 30: blue LED unit; 40: power supply unit; 50: start unit; 60: housing; 61: groove; 62: main board; 63: cover body; 64: transparent cover; 65: sliding chute; 651: buckling pin; 652: hook; 66: decorative ring; 661: buckling foot; 70: shell.

### DETAILED DESCRIPTION

The following clearly and completely describes the technical solutions in the embodiments of the present invention with reference to the accompanying drawings in the embodiments of the present invention. Apparently, the described embodiments are some of the embodiments of the present invention rather than all of the embodiments. All other embodiments obtained by a person of ordinary skill in the art based on the disclosed embodiments without creative efforts shall fall within the protection scope of the present invention.

It should be understood that when used in this specification and the appended claims, the terms “comprise” and “include” indicate the presence of described features, integers, steps, operations, elements and/or components, but do not exclude the presence or addition of one or more of other features, integers, steps, operations, elements, components, and/or combinations thereof.

It should also be understood that the terms used in the specification of the present invention are merely for the purpose of illustrating specific embodiments, and are not intended to limit the present invention. As used in the specification of the present invention and the appended claims, the singular forms “a”, “an” and “the” are intended to include the plural forms unless the context clearly indicates otherwise.

It should also be further understood that the term “and/or” as used in the specification of the present invention and the appended claims refers to any combination or all possible combinations of one or more of items listed in association, and includes these combinations.

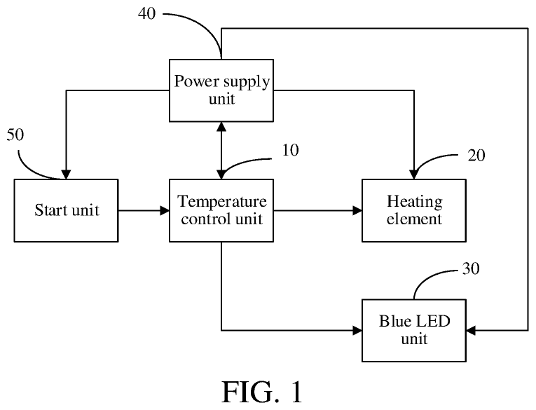

With reference to FIG. 1, FIG. 1 is a schematic block diagram of an itching relieving apparatus provided by an embodiment of the present invention; the itching relieving apparatus can be used indoors and outdoors; the temperature of a heating element 20 is accurately controlled and a stable voltage support is provided through a main control chip U4 for lossless release of blue LEDs with a wavelength of 465 nm; and in combination with the design of a face cover with high light transmittance, blue light of 465 nm and the temperature sensed by the heating element 20 are combined and effectively applied to affected parts, thereby achieving the aim of rapidly relieving itching.

With reference to FIG. 1, the aforementioned itching relieving apparatus includes a temperature control unit 10, a heating element 20 and a blue LED unit 30. The temperature control unit 10 is configured to: supply power and start the blue LED unit 30 and receive a feedback resistance coefficient corresponding to a current temperature of the heating element 20; after controlling the heating element 20 to heat up to a target temperature, power on and off the heating element 20 according to the feedback resistance coefficient to keep the temperature of the heating element 20 stable. The heating element 20 is configured to supply heat. The blue LED unit 30 is configured to start working under the power supply of the temperature control unit 10, and stabilize a corresponding power supply.

Heat generated by the heating element 20 is fed back to a thermistor R29. The aforementioned temperature control unit 10 may determine whether the heating element 20 reaches a target temperature according to a feedback resistance coefficient of the thermistor R29. If the heating element does not reach the target temperature, the heating element 20 is controlled for continued heating. If the heating element reaches the target temperature, powering-on/off operation is performed on the heating element 20 to keep the temperature of the heating element 20 stable. Further, the blue LED unit 30 can stabilize the power supply voltage provided for the blue LEDs, thereby achieving the aims of uniform and stable temperature of a heating and electric heating element, improvement of the itching relieving effect on patients, and rapid itching relieving.

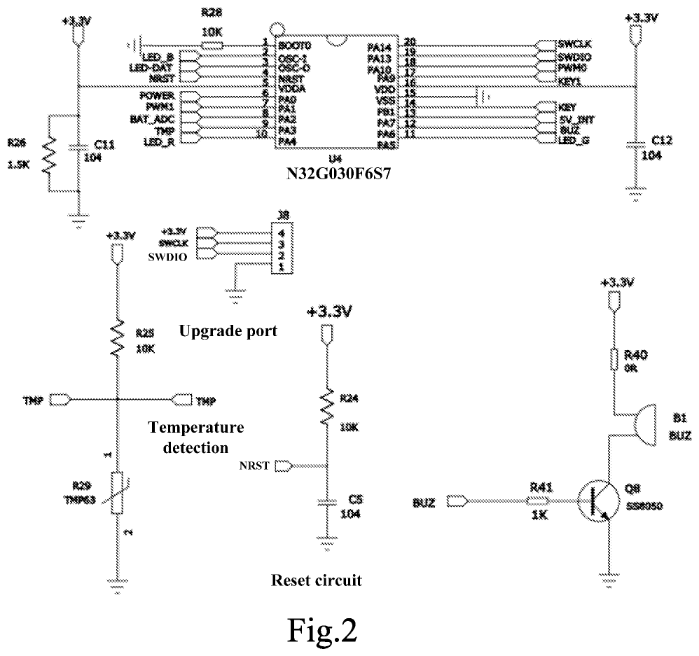

With reference to FIG. 2, the aforementioned temperature control unit 10 includes a main control chip U4, the thermistor R29, and a resistor R25. The main control chip U4 is connected to the thermistor R29 and the resistor R25, respectively. The thermistor R29 is configured to detect the current temperature of the heating element 20 and form the corresponding feedback resistance coefficient.

In this embodiment, the main control chip U4 is a G030 series chip and the model is but not limited to N32G030F6S7.

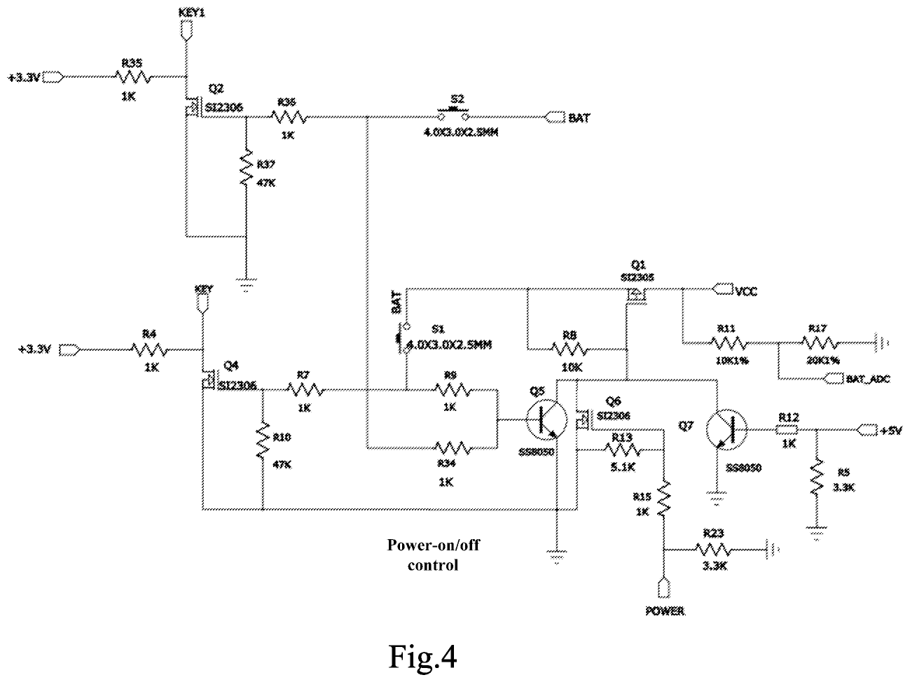

With reference to FIG. 4, the aforementioned itching relieving apparatus further includes a start unit 50. The start unit 50 includes a start key S1 and a start key S2, and the start key S1 and the start key S2 are connected to the main control chip U4.

In this embodiment, after the whole itching relieving apparatus is started through the start key S1 or the start key S2 of the start unit 50, the temperature control unit 10 receives the resistance coefficient of the thermistor R29 corresponding to the current temperature of the heating element 20 through the main control chip U4. When the resistance coefficient of R29 reaches a preset resistance corresponding to a constant temperature of the start key S1 or the start key S2, the main control chip U4 realizes the stabilization of the temperature of the heating element 20 and the resistance of the resistor R29 by rapidly switching between powering on and powering off. When the time for reaching stabilization reaches preset time of the start key S1 or the start key S2, the start unit 50, the heating element 20, and the temperature control unit 10 are powered off at the same time, thereby ensuring that the temperature of the heating and electric heating element is uniform and stable.

In this embodiment, with reference to FIG. 4, the aforementioned start key S2 is connected to the aforementioned main control chip U4 through an MOS tube Q2. The aforementioned start key S1 is connected to the aforementioned main control chip U4 through an MOS tube Q4. The MOS tube Q4 is further connected to a triode Q5 and a triode Q7, and the triode Q7 is connected to a power supply unit 40, so that the itching relieving apparatus can be started by using the start keys S1 and S2.

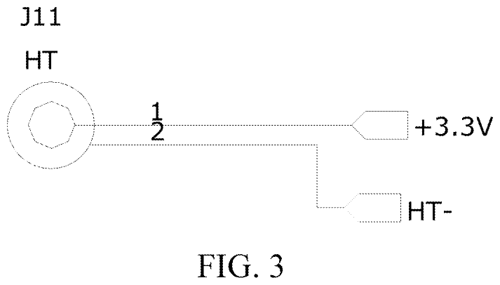

In this embodiment, with reference to FIG. 3, the aforementioned heating element 20 includes an aluminum oxide substrate heating sheet J11, and the aluminum oxide substrate heating sheet J11 is connected to the main control chip U4 through a control chip U2.

Sites needing itching relieving of the patients are heated by using the aluminum oxide substrate heating sheet J11 and heat can accelerate the blood circulation around the affected parts and accelerate the rapid metabolism of weakly acidic substances and chemicals that cause itching after mosquito bites.

In one embodiment, with reference to FIG. 4, the aforementioned itching relieving apparatus further includes a power supply unit 40. The power supply unit 40 is configured to supply power to the temperature control unit 10, the heating element 20, and the blue LED unit 30. Specifically, voltages of 5 V and 3.3 V are provided.

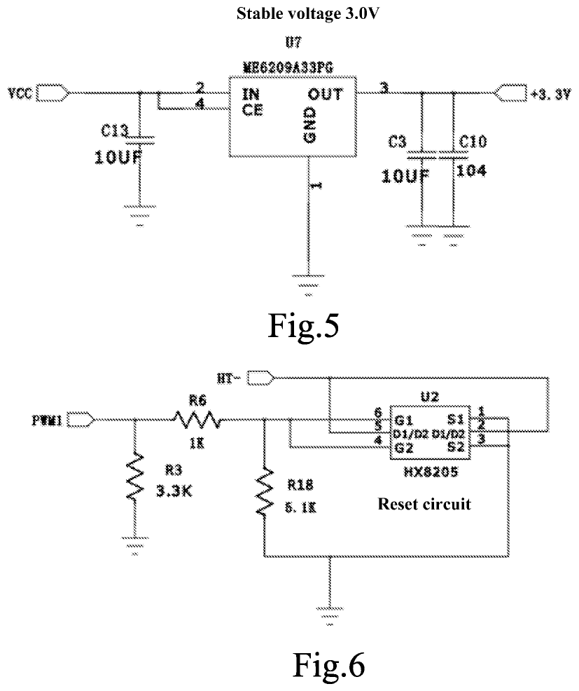

In one embodiment, with reference to FIG. 5 to FIG. 7, the aforementioned blue LED unit 30 includes a voltage regulator module, a blue light control module, and an LED blue light. The blue light control module is connected to the main control chip U4 and the power supply unit 40. respectively. The voltage regulator module is connected between the power supply unit 40 and the blue light control module.

In one embodiment, with reference to FIG. 6, the aforementioned blue light control module includes the control chip U2.

In one embodiment, with reference to FIG. 5, the aforementioned voltage regulator module includes a voltage regulator chip U7.

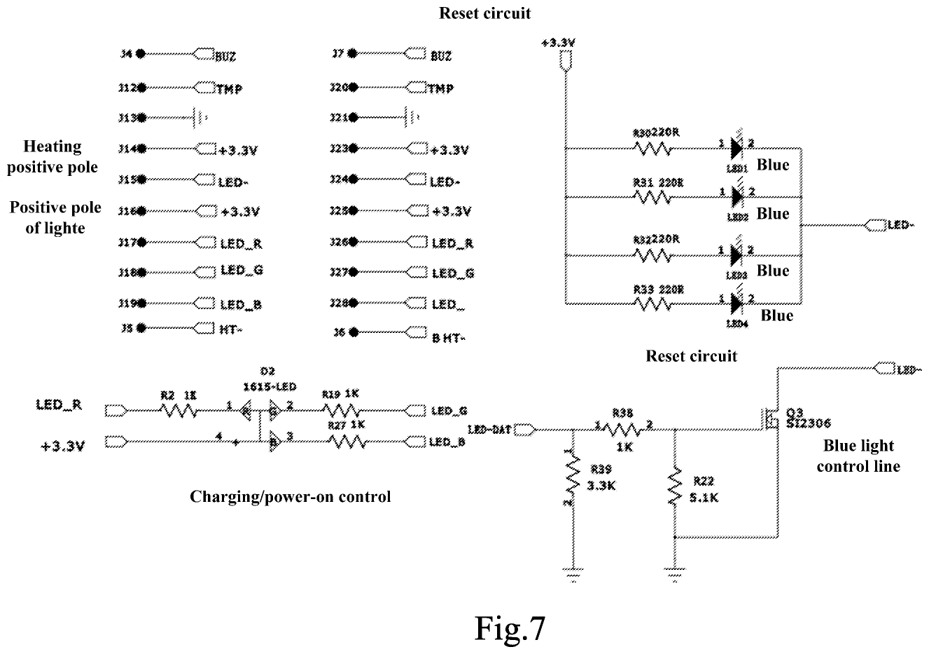

In one embodiment, with reference to FIG. 7, the aforementioned LED blue lights include LED1, LED2, LED3, and LED4. The LED1, the LED2, the LED3, and the LED4 are connected to a main control chip U1 through an MOS tube Q3.

All of these four LED blue lights are LED blue lights with the wavelength of 465 nm and have anti-inflammatory and bactericidal effects on the affected parts. The heating element 20 and the LED blue light are combined to reach the effects of rapidly relieving itching and assisting in eliminating redness and swelling.

Additionally, the itching relieving apparatus is further provided with an indicator light for indicating a charging or power-on state. The indicator light is connected to the main control chip U1 through a triode D2.

Specifically, After the itching relieving apparatus is powered on through S1 or S2 of the start unit 50, the G030 series chip supplies power to the blue LED control module through an LED-DAT pin to start. Since the cell voltage varies with power, in order to prevent the wavelength of 465 nm of the blue light LED module from wavelength fluctuation caused by voltage, the voltage regulator module is used for stabilizing the voltage to 3.3 V corresponding to the wavelength of 465 nm of the blue LEDs, thereby stabilizing the wavelength of blue light and ensuring the use effect of the product.

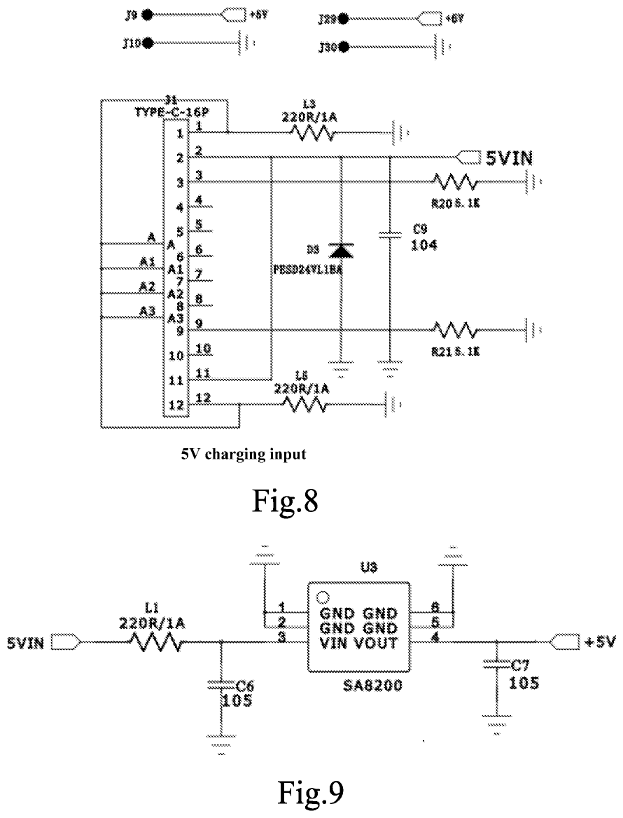

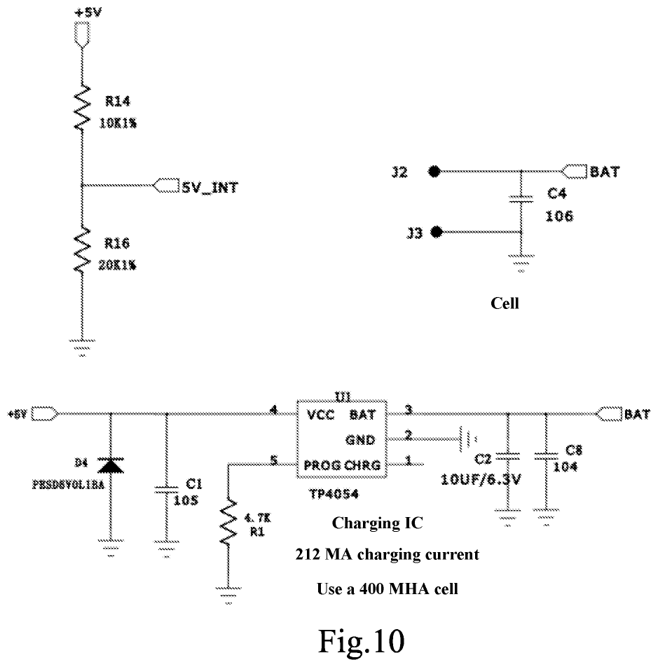

In one embodiment, the aforementioned power supply unit 40 includes a 5 V charging input module, an input protection module, and a charging IC. With reference to FIG. 8, the aforementioned 5 V charging input module includes a TYPE interface J1. With reference to FIG. 9, the aforementioned input protection module includes an input protection chip U3. The input protection chip U3 is connected to the TYPE interface J1 and the model of the input protection chip U3 is but not limited to SA8200. With reference to FIG. 10, the aforementioned charging IC includes a chip U1 connected to the input protection chip U3, and the model of the chip U1 is but not limited to TP4054.

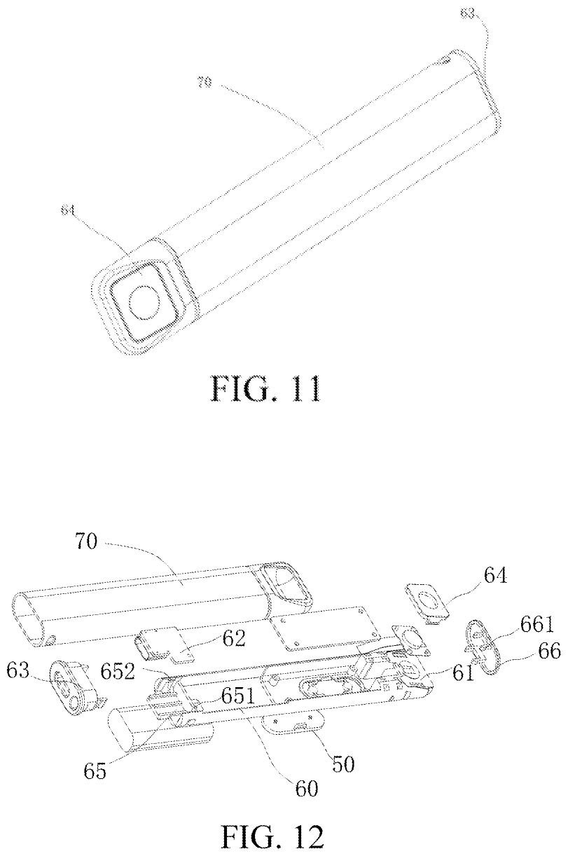

In one embodiment, with reference to FIG. 11 and FIG. 12, the aforementioned itching relieving apparatus further includes a housing 60. The housing 60 is internally provided with a main board 62, and the temperature control unit 10 is provided on the main board 62.

In this embodiment, the aforementioned housing 60 is internally provided with a sliding chute 65. A buckling pin 651 is provided on an upper side wall of the sliding chute 65. A mounting hole is formed in the main board 62. The buckling pin 651 is embedded into the mounting hole. An opening is formed in an upper end of the sliding chute 65. A hook 652 is provided on two sides of the opening, and the hook 652 is used for clamping the main board 62, realizing detachable and stable installation of the main board 62.

In one embodiment, with reference FIG. 11 and FIG. 12, an upper end face of the aforementioned housing 60 is downward recessed to form a groove 61. The LED blue light is placed in the groove 61, and a transparent cover 64 caps over the LED blue light.

In this embodiment, the transparent cover 64 is made of a PC material with high light transmittance, which makes the light transmittance reach 90% or above to ensure the effect of the blue light of 465 nm and prevents the wavelength of the blue light from loss after transmitting through the PC cover. The PC transparent cover 64 with the light transmittance of 97% is structurally designed to ensure that the wavelength is still within an effective range after transmission, and rapidly and effectively realizes the effects of relieving itching and assisting in eliminating redness and swelling under both an efficient mode and a gentle mode in combination with the above temperature control and blue light control.

In this embodiment, a buckle is provided on two sides of the aforementioned transparent cover 64 and the housing 60 is provided with a clamping slot; and the aforementioned buckle is clamped within the clamping slot, realizing detachable connection of the transparent cover 64.

In one embodiment, with reference FIG. 11 and FIG. 12, one end of the aforementioned housing 60 far away from the LED blue light is connected to a cover body 63 through a buckle structure.

The cover body 63 is provided with a through hole for a connector to penetrate through, so as to facilitate the charging of the main board 62.

In one embodiment, the aforementioned housing 60 is provided with a decorative ring 66. The decorative ring 66 is provided with buckling feet 661; and the buckling feet 661 are connected onto the housing 60 in a buckle connection manner.

In this embodiment, with reference to FIG. 2, the aforementioned main control chip U4 is further connected to a buzzer B1, reminding the end of a single course of treatment.

The aforementioned housing 60 is externally provided with a shell 70.

The aforementioned itching relieving apparatus has a temperature protection mechanism. By means of an algorithm, the efficient mode (i.e., a mode corresponding to the key S1) is accurately controlled at 51° C. and operated for 10 seconds and the mild mode (i.e., the key S2) is controlled at 47° C. and operated for 13 seconds. Accurate temperature control is realized by the efficient mode and the mild mode under the control of a software algorithm, which can ensure the effectiveness of the product. At the same time, by means of real-time induced temperature of a temperature sensing module (i.e., the thermistor R29) connected to the heating element 20 of an aluminum oxide substrate, in a case that the resistance coefficient of the temperature sensing module is abnormal due to abnormal temperature of the product under uncontrollable environments or use conditions, a signal is fed back immediately to power off the start unit 50, thereby avoiding undesirable phenomena such as scalds.

The aforementioned itching relieving apparatus is provided with the temperature control unit 10, the heating element 20, and the blue LED unit 30. The temperature of the heating element 20 is controlled in real time by using the temperature control unit 10. When the temperature of the heating element 20 reaches a target temperature, the temperature of the heating element 20 is kept stable by powering on and off within a specified time period to realize uniform and stable temperature of the heating and electric heating element and provide stable voltage for the LED blue light, thereby improving the itching relieving effect on the patients.

In one embodiment, an operating method of an itching relieving apparatus is further provided, including:

in a case that the itching relieving apparatus works, after the temperature control unit 10 controls the heating element 20 to heat up to a target temperature according to a feedback resistance coefficient corresponding to a current temperature of the heating element 20, powering on and off the heating element 20 according to the feedback resistance coefficient to keep the temperature of the heating element 20 stable; and providing stable voltage through the temperature control unit 10 and the blue LED unit 30 to ensure that the peak wavelength of blue LEDs is output without loss.

It should be noted that: Those skilled in the art can clearly understand that for the specific implementation process of the aforementioned operating method of an itching relieving apparatus, reference may be made to the corresponding descriptions in the foregoing itching relieving apparatus embodiments, and for convenience and conciseness of descriptions, details are not described herein again.

The foregoing descriptions are merely specific implementations of the present invention, but are not intended to limit the protection scope of the present invention. Any modification or replacement readily figured out by those skilled in the art within the technical scope disclosed in the present invention shall fall within the protection scope of the present invention. Therefore, the protection scope of the present invention shall be subject to the protection scope of the claims.

What is claimed is:

1. An itching relieving apparatus, comprising a temperature control unit, a heating element, and a blue LED unit; the temperature control unit being configured to: supply power and start the blue LED unit and receive a feedback resistance coefficient corresponding to a current temperature of the heating element; after controlling the heating element to heat up to a target temperature, power on and off the heating element according to the feedback resistance coefficient to keep the temperature of the heating element stable; the heating element being configured to supply heat; and the blue LED unit being configured to start working under the power supply of the temperature control unit, and stabilize a corresponding power supply.

2. The itching relieving apparatus according to claim 1, wherein the temperature control unit comprises a main control chip U4, a thermistor R29, and a resistor R25; the main control chip U4 is connected to the thermistor R29 and the resistor R25, respectively; and wherein the thermistor R29 is configured to detect the current temperature of the heating element and form the corresponding feedback resistance coefficient.

3. The itching relieving apparatus according to claim 2, wherein the heating element comprises an aluminum oxide substrate heating sheet J11, the aluminum oxide substrate heating sheet J11 being connected to the main control chip U4 through a control chip U2.

4. The itching relieving apparatus according to claim 2, further comprising a power supply unit, the power supply unit being configured to supply power to the temperature control unit, the heating element, and the blue LED unit.

5. The itching relieving apparatus according to claim 4, wherein the blue LED unit comprises a voltage regulator module, a blue light control module, and an LED blue light, the blue light control module being connected to the main control chip U4 and the power supply unit, respectively; and the voltage regulator module being connected between the power supply unit and the blue light control module.

6. The itching relieving apparatus according to claim 2, further comprising a start unit, the start unit comprising a start key S1 and a start key S2 and the start key S1 and the start key S2 being connected to the main control chip U4.

7. The itching relieving apparatus according to claim 5, further comprising a housing, the housing being internally provided with a main board and the temperature control unit being provided on the main board.

8. The itching relieving apparatus according to claim 7, wherein an upper end face of the housing is downward recessed to form a groove, the LED blue light is placed in the groove, and a transparent cover caps over the LED blue light.

9. The itching relieving apparatus according to claim 8, wherein one end of the housing far away from the LED blue light is connected to a cover body through a buckle structure.

10. An operating method of an itching relieving apparatus, comprising:
in a case that the itching relieving apparatus works, after a temperature control unit controls a heating element to heat up to a target temperature according to a feedback resistance coefficient corresponding to a current temperature of the heating element, powering on and off the heating element according to the feedback resistance coefficient to keep the temperature of the heating element stable; and providing stable voltage through the temperature control unit and a blue LED unit to ensure that a peak wavelength of blue LEDs is output without loss.
